(19) United States
(12) Patent Application Publication
(10) Pub. No.: US 2003/0204253 A1
(43) Pub. Date: Oct. 30, 2003

(54) Posterior chamber phakic lens

(76) Inventor: Anilbhai S. Patel, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/135,464

(22) Filed: Apr. 30, 2002

(51) Int. Cl. A61F 2/16

(52) U.S. Cl. 623/6.15; 623/6.49

(57) ABSTRACT

A posterior chamber phakic lens made from an elastomeric, foldable, highly biocompatible material. The lens has a generally circular optic and a pair of integrally formed haptics. The haptics project posteriorly from the optic. A plurality of holes or slots are formed along the edge of the optic and along the length of the haptics. A soft, highly biocompatible material may be placed into the holes or slots by a friction fit or a primary/secondary molding process. Such a construction helps to avoid pupillary blockage, allows for improved aqueous flow around the natural lens and provides a spacer to hold the lens away from the anterior capsular and the iris.

### BACKGROUND OF THE INVENTION

This invention relates generally to the field of intraocular lenses (IOL) and, more particularly, to posterior chamber phakic IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). Recently, the use of photablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. All of these refractive surgical procedures cause an irreversible modification to the shape of the cornea in order to effect refractive changes, and if the correct refraction is not achieved by the first procedure, a second procedure or enhancement must be performed. Additionally, the long-term stability of the correction is somewhat variable because of the variability of the biological wound healing response between patients.

Several companies are investigating implantable posterior chamber phakic IOLs, including the Staar ICL lens and the Medennium PRL lens. These and other anterior chamber phakic lenses are described in U.S. Pat. Nos. 4,769,035 (Kelman), 6,015,435 (Valunin, et al.) and 6,106,553 (Feingold), the entire contents of which being incorporated herein by reference. The clinic experience with commercially available anterior chamber phakic lenses has not been entirely satisfactory due to pupillary block, unwanted rotation of the lens, iris chafing induced pigmentary dispersion glaucoma and the development of traumatic cataract.

Therefore, a need continues to exist for a safe, stable and biocompatible posterior chamber phakic intraocular lens.

### BRIEF SUMMARY OF THE INVENTION

The present invention improves upon the prior art by providing a posterior chamber phakic lens made from an elastomeric, foldable, highly biocompatible material. The lens has a generally circular optic and a pair of integrally formed haptics. The haptics project posteriorly from the optic. A plurality of holes or slots are formed along the edge of the optic and along the length of the haptics. A soft, highly biocompatible material may be placed into the holes or slots by a friction fit or a primary/secondary molding process. Such a construction helps to avoid pupillary blockage, allows for improved aqueous flow around the natural lens and provides a spacer to hold the lens away from the anterior capsular and the iris and thus, helps to reduce the potential for induced cataract formation and induced pigmentary dispersion glaucoma, respectively.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible intraocular lens that allows for increased aqueous flow to the natural lens.

Still another objective of the present invention is to provide a safe and biocompatible intraocular lens that prevents the bulk of the lens from contacting the iris or anterior capsule.

Still another objective of the present invention is to provide a safe and biocompatible intraocular lens that reduces the potential for induced cataract formation and induced pigmentary dispersion glaucoma

These and other advantages and objectives of the present invention will become apparent from the detailed description and claims that follow.

### BRIEF DESCRIPTION OF THE DRAWING

FIG. 1 is an enlarger perspective view of the lens of the present invention.

FIG. 2 is an enlarged side view of the lens of the present invention.

FIG. 3 is an enlarged bottom plan view of the lens of the present invention.

### DETAILED DESCRIPTION OF THE INVENTION

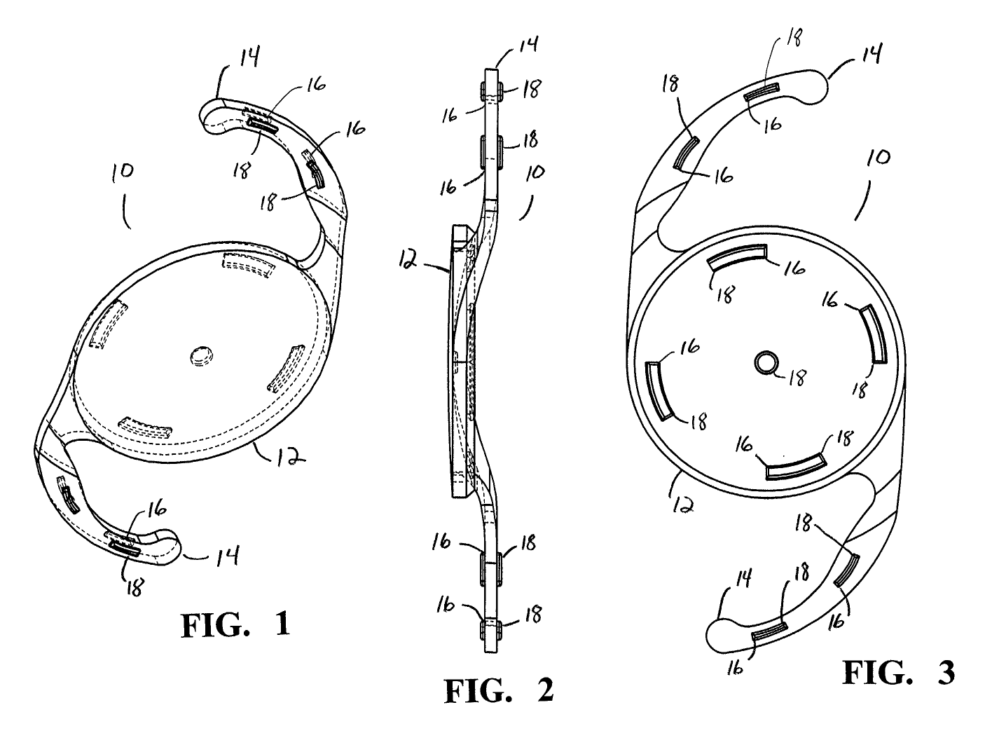

As best seen in FIGS. 1, 2 and 3, lens 10 of the present invention generally includes optic 12 and at least two haptics 14 integrally formed with optic 12. Optic 12 may be of any suitable size, such as between 4.5 mm and 6.5 mm in diameter, and may be biconcave, biconvex, concave/convex or any other suitable geometry. Optic 12 may also contain refractive or diffractive features, such features being well-known in the art. Lens 10 is preferably formed in any suitable overall length, for example, around 12 millimeters, for implantation in the posterior chamber in front of the natural lens from a soft, foldable material such as a hydrogel, silicone or soft acrylic, such diameters and materials being well-known in the art. As best seen in FIG. 3, haptics 14 project or vault posteriorly from optic 12, so as to locate optic 12 anteriorly of haptics 14 once implanted in an eye. Formed along haptics 14 and/or optic 12 are a plurality of holes or slots 16. Inserted into holes 16, either through mechanical means or by a primary or secondary molding operation, are a plurality of spacers 18. Spacers 18 project approximately between 0.05 millimeters to 0.3 millimeters from optic 12 and haptics 14 an d are made from a soft, highly biocompatible materials such as a high (greater than about 45%) water content hydrogel material. Preferably, spacers project at least anteriorly from haptics 14 and at least posteriorly from optic 12, but spacers 18 may project both anteriorly and posteriorly from both optic 12 and haptics 14.

This description is given for purposes of illustration and explanation. It will be apparent to those skilled in the relevant art that changes and modifications may be made to the invention described above without departing from its scope or spirit.

I claim:

1. An intraocular lens, comprising:
a) an optic;
b) at least two haptics connected to the optic, the haptics containing a plurality of holes; and
c) a plurality of spacers contained within the holes, the spacers projecting outwardly from the haptics.

2. The lens of claim 1 wherein the haptics are integrally formed with the optic.

3. The lens of claim 1 wherein the optic comprises a soft acrylic.

4. The lens of claim 1 wherein the optic comprises a hydrogel.

5. The lens of claim 2 wherein the lens comprises a soft acrylic.

6. The lens of claim 2 wherein the lens comprises a hydrogel.

7. The lens of claim 1 wherein the spacers comprise a hydrogel.

8. An intraocular lens, comprising:
a) an optic containing a plurality of holes;
b) at least two haptics connected to the optic; and
c) a plurality of spacers contained within the holes, the spacers projecting outwardly from the optic.

9. The lens of claim 8 wherein the haptics are integrally formed with the optic.

10. The lens of claim 8 wherein the optic comprises a soft acrylic.

11. The lens of claim 8 wherein the optic comprises a hydrogel.

12. The lens of claim 9 wherein the lens comprises a soft acrylic.

13. The lens of claim 9 wherein the lens comprises a hydrogel.

14. The lens of claim 8 wherein the spacers comprise a hydrogel.

15. An intraocular lens, comprising:
a) an optic containing a plurality of first holes;
b) at least two haptics connected to the optic, the haptics containing a plurality of second holes; and
c) a plurality of spacers contained within the first and the second holes, the spacers projecting outwardly from the optic and the haptics.

16. The lens of claim 15 wherein the haptics are integrally formed with the optic.

17. The lens of claim 15 wherein the optic comprises a soft acrylic.

18. The lens of claim 15 wherein the optic comprises a hydrogel.

19. The lens of claim 16 wherein the lens comprises a soft acrylic.

20. The lens of claim 16 wherein the lens comprises a hydrogel.

21. The lens of claim 15 wherein the spacers comprise a hydrogel.
